(19) United States
(12) Patent Application Publication
(10) Pub. No.: US 2026/0219186 A1
(43) Pub. Date: Jul. 30, 2026

(54) SYSTEMS FOR UPCONVERSION CHEMILUMINESCENCE DETECTION BASED ON MAGNETIC MICROPARTICLES

(71) Applicant: SUZHOU HELMEN PRECISION INSTRUMENTS CO., LTD., Suzhou (CN)

(72) Inventors: Kai JIANG, Suzhou (CN); Tao ZHANG, Suzhou (CN); Ping WANG, Suzhou (CN); Yang WANG, Suzhou (CN); Yawei TANG, Suzhou (CN)

(73) Assignee: SUZHOU HELMEN PRECISION INSTRUMENTS CO., LTD., Suzhou (CN)

(21) Appl. No.: 19/575,893

(22) Filed: Mar. 23, 2026

Related U.S. Application Data
(63) Continuation of application No. PCT/CN2024/096778, filed on May 31, 2024.

(30) Foreign Application Priority Data
Sep. 22, 2023 (CN) 202311230383.7

(51) Int. Cl. G01N 21/64; G01N 27/74; G01N 35/04

(52) U.S. Cl. CPC G01N 21/6428; G01N 21/6445; G01N 27/745; G01N 35/04; G01N 2035/0465

(57) ABSTRACT

Disclosed is a system for upconversion chemiluminescence detection based on magnetic microparticles. A sample carrying module is disposed on a working surface and configured to input, preload, and output a plurality of samples. A gripping module is configured to grip a reaction cup and a pipette tip. A sample aspiration and dispensing module is configured to aspirate one of the plurality of samples and reagents and dispense one of the plurality of samples and the reagents into the reaction cup. The reagents include at least a monoclonal antibody reagent bound with magnetic microparticles and a monoclonal antibody reagent labeled with an up-converting phosphor. A detection preparation module is configured to perform one or more cycles of mixing, incubation, and washing and separation on the sample and the reagent. An optical measurement module is configured to excite an up-converting luminescent label and detect a fluorescence signal intensity.

### CROSS-REFERENCE TO RELATED APPLICATIONS

This application is a Continuation of International Application No. PCT/CN2024/096778, filed on May 31, 2024, which claims priority to Chinese Patent Application No. 202311230383.7, filed on Sep. 22, 2023, the entire contents of each of which are hereby incorporated by reference.

### TECHNICAL FIELD

The present disclosure generally relates to the field of testing or analyzing materials by measuring chemical or physical properties of the materials, in particular to a system for upconversion chemiluminescence detection based on magnetic microparticles.

### BACKGROUND

A magnetic microparticle chemiluminescence method is currently the most important immunoassay technology in the field of in vitro diagnostics (IVD). The most important detection equipment in hospital clinical laboratories is a fully automatic magnetic microparticle chemiluminescence detection apparatus. The demand for reagents and instruments each year is extremely high and continues to increase year by year.

Currently, magnetic microparticle chemiluminescence mainly includes three subdivided methodologies, which are electrochemiluminescence, acridinium ester direct chemiluminescence, and enzyme-catalyzed indirect chemiluminescence. Electrochemiluminescence is an exclusive patented technology of Roche. It uses electrical energy to excite labels into a high-energy unstable state, which then emits fluorescence via electron transition back to the ground state. The drawback of this methodology is that the electrodes applying the electric energy must be in direct contact with the sample solution, which means extremely high requirements for electrode cleaning and a high risk of cross-interference. For this reason, not many manufacturers are currently developing electrochemiluminescence technologies. Both acridinium ester direct chemiluminescence and enzyme-catalyzed indirect chemiluminescence utilize independent reaction cups to isolate samples from each other, thereby preventing cross-interference and eliminating the risk of cross-contamination. A trigger solution is used to catalyze acridinium ester, or alkaline phosphatase (horseradish peroxidase) catalyzes a matrix to initiate a chemical reaction. This excites outer electrons of a luminescent label to a high-energy unstable state, which then transition to the ground state and emit fluorescence. However, these methods of luminescence all rely on biochemical reactions to generate excitation energy, which is generally weak and results in low luminescence signal intensity. In addition, bioluminescence is a one-time event and cannot be repeatedly excited or detected.

Therefore, the present disclosure provides a system for upconversion chemiluminescence detection based on magnetic microparticles to solve the problems of cross-interference, weak fluorescence signal intensity, and inability to perform repeated detections.

### SUMMARY

The technical concept of one or more embodiments of the present disclosure is that materials composed of certain rare-earth metal elements doped into a crystal lattice exhibit up-converting luminescence. There are three main components in the materials: a host matrix, an absorber, and an emitter. Crystal materials serving as the host matrix include oxysulfides (e.g., Y₂O₂S, GdO₂S, La₂O₂S, etc.), fluorides (e.g., YF₃, GdF₃, LaF₃, etc.), gallates (e.g., YGaO₃, Y₃Ga₅O₁₂, etc.), silicates (e.g., YSi₂O₅, YSi₃O₇, etc.), etc. Rare-earth metal ions commonly used as the absorber include ytterbium ions (Yb³⁺), erbium ions (Er³⁺), samarium ions (Sm³⁺), etc. Rare-earth metal ions commonly used as the emitter include erbium ions (Er³⁺), holmium ions (Ho³⁺), thulium ions (Tm³⁺), terbium ions (Tb³⁺). A suitable spatial orientation and distance of an absorber-emitter ion pair within the host matrix lattice are the basis for generating up-converting luminescence. The generation of up-converting luminescence is an optical process involving a plurality of photons (at least two photons). In this process, an absorber (e.g., Yb³⁺) within an up-converting phosphor must absorb at least two low-energy photons (in an infrared region, e.g., 980 nm). Then after a series of internal energy conversions, the absorber transmits the energy of the two photons consecutively to an emitter (e.g., Er³⁺) in a non-radiative form (A1→A2, A2→A3), thereby placing the emitter in an excited state (A3). The emitter then undergoes a transition back to a ground state energy level, releasing a high-energy photon (in a visible region, e.g., 525 nm or 540 nm) to complete energy upconversion.

The up-converting phosphor (UCP) serves as a label for magnetic microparticle chemiluminescence. Compared with traditional bioluminescence technology, the UCP has the advantage of being capable of repeated excitations. Furthermore, excitation energy from a high-power laser far exceeds the biological energy of bioluminescence, leading to a substantial increase in fluorescence signal intensity. The wavelength of an excitation light source is 980 nm. Apart from the UPC, the excitation light source generally cannot excite fluorescence of other wavelengths from other consumables or optical component materials used in the detection reaction. Accordingly, the background fluorescence signals are very clean, which is beneficial for improving detection sensitivity. Compared with traditional down-converting fluorescent labels (e.g., quantum dots or Cy5 fluorescent dyes), UCP labels have the advantage of clean background fluorescence signals. Consumables such as a reaction cup typically contain trace amounts of down-converting phosphor, which can contaminate the background signals of fluorescence detection, resulting in inaccurate detection. However, in up-converting fluorescence detection, the excitation light source is infrared excitation, which does not excite other fluorescence signals, further improving sensitivity.

One or more embodiments of the present disclosure provide a system for upconversion chemiluminescence detection based on magnetic microparticles. The system includes a working surface. The working surface includes: a sample carrying module configured to input, preload, and output a plurality of samples; a gripping module configured to grip a reaction cup and a pipette tip (hereinafter also referred to as a tip) from a previous working position to a next working position; a sample aspiration and dispensing module configured to aspirate at least one of the plurality of samples and reagents and dispense the at least one of the plurality of samples and the reagents into the reaction cup, the reagents including at least a monoclonal antibody reagent bound with magnetic microparticles and a monoclonal antibody reagent labeled with up-converting phosphor; a detection preparation module configured to perform one or more cycles of mixing, incubation, and a plurality of cycles of washing and separation on the at least one of the plurality of samples and the reagents dispensed into the reaction cup; and an optical measurement module configured to excite an up-converting luminescent label and detect a fluorescence signal intensity.

In some embodiments, the sample carrying module includes a sample loading channel and a sample output channel. The sample loading channel is provided with a sample inlet for placing a sample rack. The sample output channel is provided with a sample outlet for outputting the sample rack. The sample loading channel and the sample output channel are respectively provided with a traction unit for the sample rack. A moving frame is respectively provided at ends of the sample loading channel and the sample output channel for the sample rack, and the moving frame is provided with a moving traction unit. A cap removal position is provided between the sample loading channel and the sample output channel, and a cap removal mechanism is provided corresponding to the cap removal position. The sample loading channel is provided with rollers tangent to a sample tube, and a side of the cap removal position is provided with a barcode scanning mechanism for the sample tube. The sample loading channel is provided with a sample position, and the sample position is configured to cooperate with the sample aspiration and dispensing module.

In some embodiments, the gripping module and the sample aspiration and dispensing module are provided with a transfer assistance module. The transfer assistance module is configured to perform transfer assistance for a travel path between the gripping module and the sample aspiration and dispensing module. The transfer assistance module includes a moving member provided with a plurality of hole positions, the moving member is disposed on a track, and the track is disposed between the gripping module and the sample aspiration and dispensing module.

In some embodiments, the gripping module includes a three-axis motion mechanism disposed above the working surface. A gripping mechanism is provided at a lower portion of a Z-axis of the three-axis motion mechanism, and the gripping mechanism is configured to cooperate with the transfer assistance module. The working surface of the gripping mechanism is provided with a tip plate for placing the pipette tip and the detection preparation module for placing the reaction cup.

In some embodiments, the sample aspiration and dispensing module includes a dual-axis motion mechanism disposed above the working surface. The dual-axis motion mechanism is provided with a first liquid aspiration mechanism and a second liquid aspiration mechanism. The first liquid aspiration mechanism is configured to pick up the pipette tip and aspirate a sample liquid from the sample position of the sample loading channel to the reaction cup. The second liquid aspiration mechanism is configured to aspirate the reagents to the reaction cup. The first liquid aspiration mechanism and the second liquid aspiration mechanism are configured to cooperate with the transfer assistance module. The second liquid aspiration mechanism is provided with a reagent storage chamber.

In some embodiments, the reagent storage chamber includes a storage chamber cavity. A rotation mechanism is provided at a bottom of the storage chamber cavity. A plurality of reagent sets are radially disposed in the storage chamber cavity. Any of the plurality of reagent sets includes a reagent tube which contains the monoclonal antibody reagent bound with the magnetic microparticles and the monoclonal antibody reagent labeled with the up-converting phosphor.

In some embodiments, the detection preparation module includes an incubation unit, a vibration and mixing unit, and a washing and separation unit. The washing and separation unit includes a rotary disk, the rotary disk is provided with a position 0 and at least three sets of washing positions. Each set of the at least three sets of washing positions includes a liquid withdrawal position and a liquid injection position. A last liquid injection position is a pre-excitation position. An outer wall of the rotary disk is provided with a permanent magnet.

In some embodiments, the optical measurement module includes a detection position. The detection position is configured to cooperate with a measurement chamber through an absorption-type movable drawer. A hollowed section is provided in a portion of the absorption-type movable drawer corresponding to a lower portion of the reaction cup. A photodetector and an excitation light source are provided in the measurement chamber corresponding to the hollowed section. A dichroic beam splitter is provided between the photodetector and the hollowed section. An incident surface of the dichroic beam splitter faces the excitation light source, a reflective surface of the dichroic beam splitter faces the hollowed section, and a transmissive surface of the dichroic beam splitter faces the photodetector.

In some embodiments, a detection method of the system includes the following operations: placing the plurality of samples in a sample rack, and introducing the plurality of samples into the system through the sample carrying module; scanning an identification number of an unprocessed sample tube in a first position, downloading a test item through the identification number, automatically uncapping the sample tube, and during a process of automatically uncapping the sample tube, gripping, by the gripping module, the reaction cup and the pipette tip to a preset position; loading, by the sample aspiration and dispensing module, the pipette tip, aspirating a fixed amount of sample, and adding the fixed amount of sample to the reaction cup; aspirating, by the sample aspiration and dispensing module based on obtained information of the test item, the monoclonal antibody reagent bound with the magnetic microparticles and the monoclonal antibody reagent labeled with the up-converting phosphor, respectively, adding the monoclonal antibody reagent bound with the magnetic microparticles and the monoclonal antibody reagent labeled with the up-converting phosphor to the reaction cup containing the fixed amount of sample, and performing a double-antibody sandwich immunoassay or a competitive immunoassay; waiting, by the sample aspiration and dispensing module, for a next sample tube to perform sample addition; gripping, by the gripping module, the reaction cup to which the fixed amount of sample and the reagents are added, transferring the reaction cup to the detection preparation module, performing shaking and mixing followed by incubating at a constant temperature for a preset time, and performing washing and separation; during a process of washing and separation, adsorbing, by the permanent magnet, the magnetic microparticles to an inner wall of the reaction cup, washing away unbound monoclonal antibody reagent labeled with the up-converting phosphor until only a complex of the monoclonal antibody reagent bound with the magnetic microparticles, an antigen of the fixed amount of sample, and the monoclonal antibody reagent labeled with the up-converting phosphor remains in the reaction cup; and transferring the reaction cup to the optical measurement module, exciting, by the optical measurement module using the excitation light source, the up-converting luminescent label, detecting a fluorescence signal intensity at a preset wavelength, and analyzing and calculating a concentration of an analyte based on the fluorescence signal intensity.

In some embodiments, after a plurality of cycles of washing and separation are completed based on the magnetic microparticles, a preset amount of diluent is added to the reaction cup after last separation, an initial mixture containing magnetic beads and the up-converting luminescent label is introduced into the optical measurement module, the initial mixture is excited through a laser, a fluorescence signal is detected recorded as X₁, the concentration of the analyte is calculated through a standard curve to obtain a target concentration Y₁. A preset amount of buffer solution is pumped into the reaction cup to dilute a reaction system to a concentration a₁, a fluorescence signal X₂ is measured again, and a target concentration Y₂ is calculated. The preset amount of buffer solution is pumped into the reaction cup again to dilute the reaction system to a concentration a₂ of an original concentration, a fluorescence signal X₃ is measured again, and a target concentration Y₃ is calculated. The preset amount of buffer solution is pumped into the reaction cup again to dilute the reaction system to a concentration a₃ of the original concentration, a fluorescence signal X₄ is measured again, and a target concentration Y₄ is calculated. The preset amount of buffer solution is pumped into the reaction cup again to dilute the reaction system to a concentration a₄ of the original concentration, a fluorescence signal X₅ is measured again, and a target concentration Y₅ is calculated. Given a target concentration value Y, a least squares formula is established, Z=(Y−Y₁)²+(a₁×Y−Y₂)²+(a₂×Y−Y₃)²+(a₃×Y−Y₄)²+(a₄×Y−Y₅)². A Y value is obtained via the least squares formula such that a Z value is a minimum value, wherein Y is an optimal target concentration value of the analyte after eliminating a magnetic bead shielding effect.

In some embodiments, the system further includes a processor. The sample aspiration and dispensing module includes a fully automatic syringe pump with a preset precision. The processor is configured to: obtain a plurality of fluorescence signal intensities obtained in a current detection; generate a response slope between the plurality of fluorescence signal intensities and a plurality of concentrations of the analyte based on the plurality of fluorescence signal intensities; and in response to the response slope being within an inconsistent interval, control the fully automatic syringe pump to dynamically change a count of steps to adjust a dynamic dilution ratio for a next detection.

In some embodiments, the processor is further configured to: after a first liquid aspiration mechanism completes an N-th proportional dilution, calculate a dilution coefficient; and in response to a difference between the dilution coefficient and a preset dilution ratio of the N-th proportional dilution exceeding a consistency threshold, control a moving member to carry the reaction cup back to a sample position to trigger the first liquid aspiration mechanism to perform a next dilution based on the dynamic dilution ratio. The consistency threshold is determined through a threshold model based on a magnetic bead feature, a buffer solution feature, and a luminescent material feature; and the threshold model is a machine learning model.

In some embodiments, the processor is further configured to: determine an optimized least squares formula based on a plurality of dynamic dilution ratios and a convergence coefficient. The convergence coefficient is determined based on a dynamic dilution difference and a consistency difference.

In some embodiments, the excitation light source is further configured to output a pulsed laser. The processor is further configured to: obtain a fluorescence decay lifetime; correct a magnetic bead shielding coefficient of magnetic beads by comparing deviation degrees of fluorescence decay lifetimes under a plurality of dynamic dilution ratios; and optimize the dynamic dilution ratio for the next detection based on the magnetic bead shielding coefficient and the response slope.

In some embodiments, a rotating assembly is provided at a detection position, and the rotating assembly is configured to drive the reaction cup to rotate at a preset speed. The processor is further configured to: obtain a signal intensity sequence of a rotating reaction cup; determine a distribution uniformity of the magnetic beads within the rotating reaction cup based on the signal intensity sequence; and in response to the distribution uniformity exceeding a uniformity threshold, issue an instruction to control a moving member to drive the reaction cup to shake again.

In some embodiments, the processor is further configured to: determine a fluorescence signal fluctuation rate based on the signal intensity sequence; determine a fluctuation weight for the current detection based on the fluorescence signal fluctuation rate; and determine an optimal target concentration value of the analyte based on the fluctuation weight and the plurality of fluorescence signal intensities.

### BRIEF DESCRIPTION OF THE DRAWINGS

FIG. 1 is a front view of a system for upconversion chemiluminescence detection based on magnetic microparticles according to some embodiments of the present disclosure;

FIG. 2 is a perspective view of a system for upconversion chemiluminescence detection based on magnetic microparticles with a portion of a housing removed according to some embodiments of the present disclosure;

FIG. 3 is a top view of FIG. 2;

FIG. 4 is a side view of FIG. 2;

FIG. 5 is a schematic structural diagram of an optical measurement module according to some embodiments of the present disclosure;

FIG. 6 is a sectional view along an A-A direction of FIG. 5.

### DETAILED DESCRIPTION

The present disclosure is further described in detail below in combination with embodiments. However, a protection scope of the present disclosure is not limited thereto.

Some embodiments of the present disclosure provide a system for upconversion chemiluminescence detection based on magnetic microparticles. It should be noted that mechanisms involving X-axis and Y-axis translation and Z-axis vertical movement in the following embodiments generally adopt a structure where a motor outputs power to a driving wheel, and the driving wheel drives a driven wheel through a synchronous belt. Those skilled in the art may also use existing transmission mechanisms for implementation, which is a conventional technique in the art.

The system provided by the embodiments of the present disclosure is described in detail below. A main working surface is defined as an XOY coordinate plane. A left-right direction is defined as an X-axis direction. A front-rear direction is defined as a Y-axis direction. A structural arrangement in the embodiments of the present disclosure may be adjusted and is not limited to the X-axis and Y-axis directions in the embodiments.

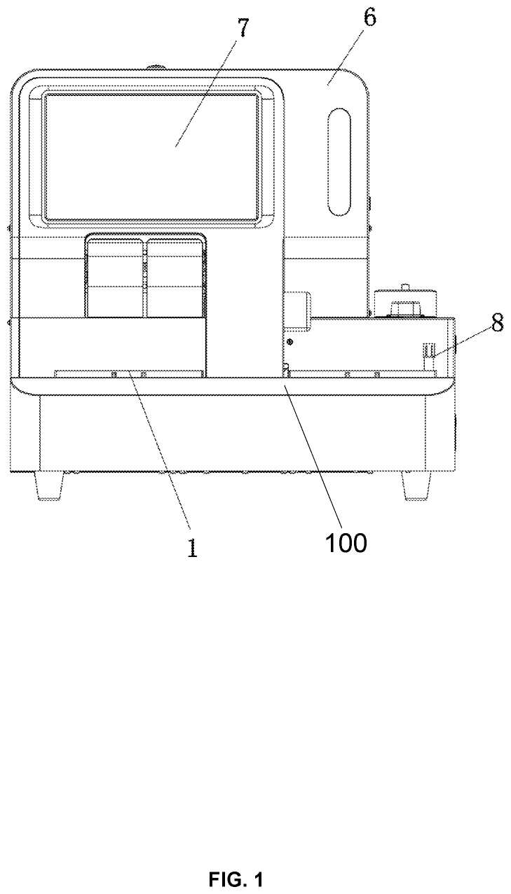

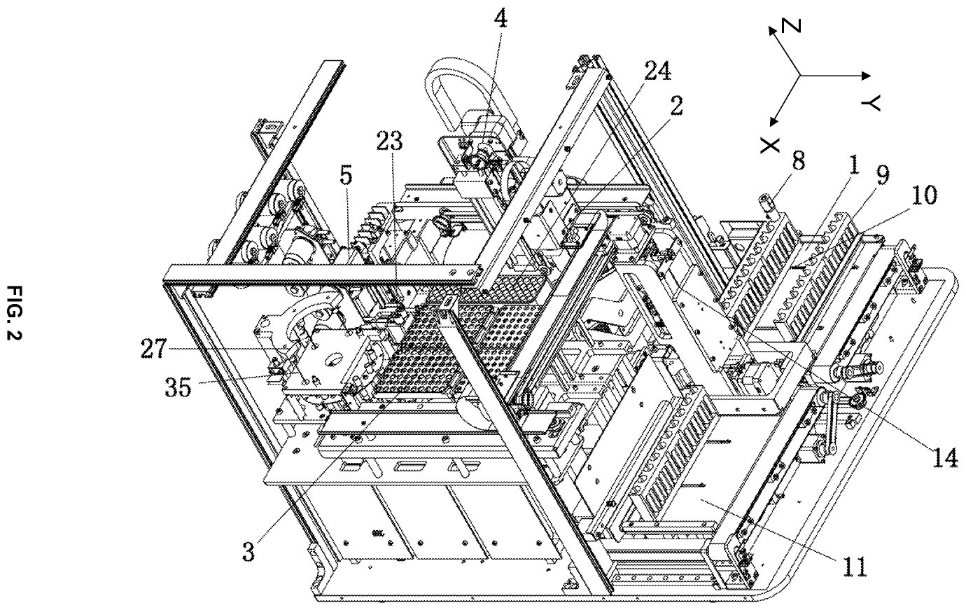

FIG. 1 is a front view of a system for upconversion chemiluminescence detection based on magnetic microparticles according to some embodiments of the present disclosure. FIG. 2 is a perspective view of a system for upconversion chemiluminescence detection based on magnetic microparticles with a portion of a housing removed according to some embodiments of the present disclosure.

As shown in FIGS. 1-2, the system includes a working surface 100. The working surface 100 is provided with a sample carrying module 1 configured to input, preload, and output a plurality of samples; a gripping module 2 configured to grip a reaction cup and a tip from a previous working position to a next working position; a tip plate 3 configured to place the reaction cup and the pipette tip; a sample aspiration and dispensing module 4 configured to aspirate at least one of the plurality of samples and reagents and disperse the at least one of the plurality of samples and the reagents into the reaction cup, the reagents at least including a monoclonal antibody reagent bound with magnetic microparticles and a monoclonal antibody reagent labeled with a UCP; a detection preparation module configured to perform one or more cycles of mixing, incubation, and a plurality of cycles of washing and separation on the at least one of the plurality of samples and the reagents dispersed into the reaction cup; and an optical measurement module 5 configured to excite an up-converting luminescent label and detect a fluorescence signal intensity.

The working surface 100 is a reference platform or a mounting substrate that carries and integrates various functional modules in the system. The working surface 100 can provide spatial positioning, structural support, and a physical interface for collaborative work for various components of the system.

In some embodiments, all modules of the system are controlled and coordinated by a controller. Those skilled in the art may set the controller according to requirements. A workflow of the controller includes: loading samples through the sample carrying module 1, griping the reaction cup and the tip by the gripping module 2, attaching the tip and then adding the samples and the reagents by the sample aspiration and dispensing module 4, completing mixing, constant-temperature incubation, and magnetic microparticle separation and washing by the detection preparation module, and finally transferring to the optical measurement module 5 to obtain a concentration of an analyte by detecting an intensity of excited fluorescence.

In some embodiments, the working surface 100 and modules on the working surface 100 are provided with a housing 6 of the system. A front side of the housing is provided with a control panel 7 and a display screen to facilitate operation for an operator. Meanwhile, an area at the sample carrying module 1 is set as an open area to facilitate loading and unloading of a sample rack 9.

In some embodiments of the present disclosure, the UCP is adopted as an up-converting fluorescent label. In an independent reaction cup, unbound up-converting fluorescent labels are washed by adopting a magnetic microparticle solid-phase labeled antibody binding and separation technique. The UCP material is excited to emit fluorescence by a high-power near-infrared laser, thereby achieving high-precision detection.

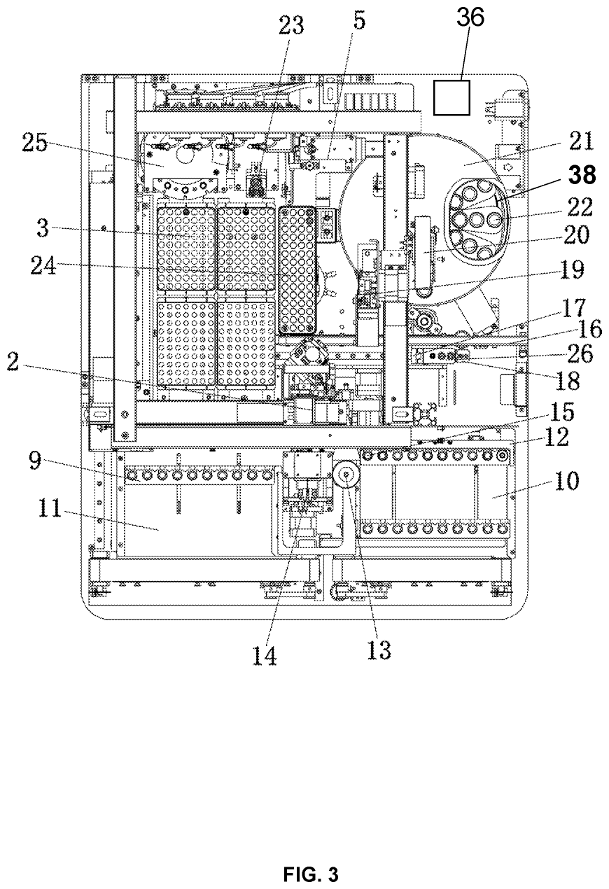

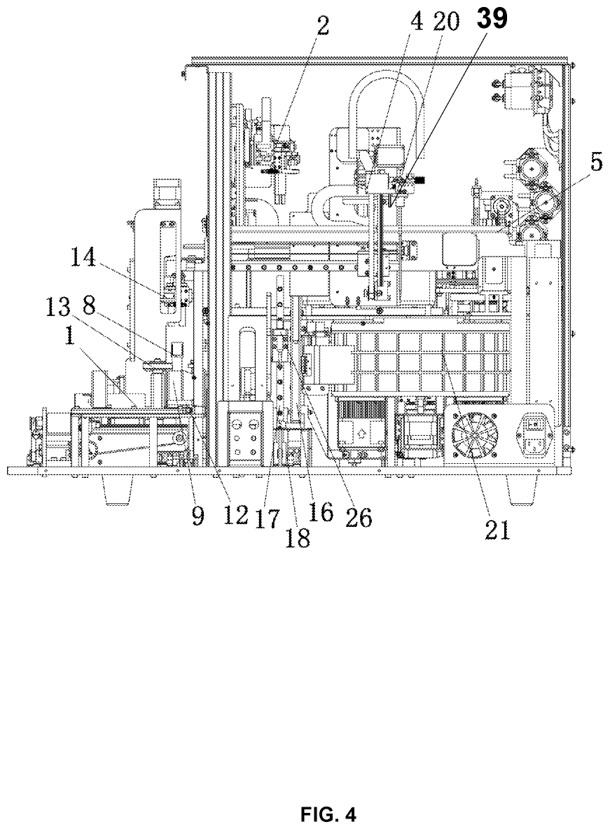

FIG. 3 is a top view of FIG. 2. FIG. 4 is a side view of FIG. 2.

In some embodiments, as shown in FIGS. 2-4, the sample carrying module 1 includes a sample loading channel 10 and a sample output channel 11. The sample loading channel 10 may be provided with a sample inlet for placing the sample rack 9. The sample output channel 11 may be provided with a sample outlet for outputting the sample rack 9. The sample loading channel 10 and the sample output channel 11 may be respectively provided with a traction unit for the sample rack 9. A moving frame 12 may be respectively provided at ends of the sample loading channel 10 and the sample output channel 11 for the sample rack 9. The moving frame 12 may be provided with a moving traction unit. A cap removal position may be provided between the sample loading channel 10 and the sample output channel 11. A cap removal mechanism 14 may be provided corresponding to the cap removal position. The sample loading channel 10 may be provided with rollers 13 tangent to a sample tube 8. A side of the cap removal position may be provided with a barcode scanning mechanism (not shown in the figure) for the sample tube 8. The sample loading channel 10 may be provided with a sample position 15. The sample position 15 may be configured to cooperate with the sample aspiration and dispensing module 4.

In some embodiments, the sample carrying module 1 implements transferring of samples and has functions such as barcode identification and uncapping. The sample loading channel 10 and the sample output channel 11 of the sample carrying module 1 may be respectively arranged on two sides of a front end of the working surface 100 and placed along an X-axis. In practical applications, the sample loading channel 10 can load up to 5 sample racks 9 at a time. Taking each sample rack 9 holding 10 samples as an example, there are 50 samples in total. A volume of each sample tube 8 may be in a range of 200 uL to 2000 uL.

In some embodiments, taking the sample loading channel 10 as an example, the sample loading channel 10 is sequentially provided with an input conveyor belt along the X-axis, an input queue area along the Y-axis, and a moving frame traction area. The moving frame traction area may match the sample output channel 11 and may be configured to move samples for which sampling is completed to the sample output channel 11 and output the samples. The sample output channel 11 may be symmetrically arranged with the sample loading channel 10.

The input conveyor belt is a conventional technique in the art. Those skilled in the art may design the input conveyor belt according to requirements or omit the input conveyor belt. When the input conveyor belt is provided, the sample rack 9 is preset to be a stop position when the sample rack 9 is aligned with the input queue area. The input queue area is provided with a panel corresponding to the input queue area along the Y-axis. The panel is provided with at least one long hole for passing through the traction unit. The traction unit here includes traction buckles respectively passing through the at least one long hole. The traction buckles are buckled with a bottom of the sample rack 9 to drive the sample rack 9 to move. The traction buckles are connected to a synchronous belt of a synchronous transmission mechanism through connection blocks. The synchronous transmission mechanism is a conventional technique in the art and includes a motor, a driving wheel provided at an output end of the motor, a driven wheel cooperating with the driving wheel, and a synchronous belt provided outside the driving wheel and the driven wheel.

The moving frame traction area may be located at an end of the input queue area. When the sample rack 9 reaches the moving frame traction area, the sample rack 9 directly enters a position of the moving frame 12. The moving traction unit is configured as a transmission mechanism of an X-axis direction. The transmission mechanism enables the moving frame 12 to drive the sample rack 9 to move forward in an X-axis range to the cap removal position (also a scanning position), move backward to the sample position 15, and after processing all samples in a current sample rack 9, move the sample rack 9 as a whole to the sample output channel 11. A structure of the sample output channel 11 is symmetrical to a structure of the sample loading channel 10 and is sequentially provided with a moving frame traction area, an output queue area along the Y-axis, and an output conveyor belt along the X-axis. A movement direction of the output conveyor belt along the X-axis is opposite to a movement direction of the input conveyor belt along the X-axis. A movement direction of the output queue area along the Y-axis is opposite to a movement direction of the input queue area along the Y-axis.

In some embodiments, the cap removal position and the scanning position are consistent. During a forward movement of the sample rack 9, the rollers 13 are arranged to be tangent to the sample tube 8 to drive the sample tube 8 to perform circular rolling. The sample tube 8 is moved to orient a barcode toward the barcode scanning mechanism by the rollers 13. The barcode scanning mechanism is configured to scan the rolling sample tube 8 in real time to obtain sample information, enabling scanning of the sample tube 8 in random placement. While obtaining the sample information through scanning, uncapping is performed by the cap removal mechanism 14. The cap removal mechanism 14 may include a descending component and a gripping component, such as a manipulator. Those skilled in the art may set the cap removal mechanism 14 according to requirements.

In some embodiments, to facilitate internal arrangement of the system, the sample position 15 cooperates with the sample aspiration and dispensing module 4. The sample position 15 cooperating with the sample aspiration and dispensing module 4 means that the sample aspiration and dispensing module 4 performs movement along the Y-axis on an XOY coordinate system. Accordingly, the sample position 15 is located directly in front of the sample aspiration and dispensing module 4.

In some embodiments, as shown in FIGS. 2-4, the gripping module 2 and the sample aspiration and dispensing module 4 are provided with a transfer assistance module. The transfer assistance module may be configured to perform transfer assistance for a travel path between the gripping module 2 and the sample aspiration and dispensing module 4. The transfer assistance module may include a moving member 26 provided with a plurality of hole positions. The moving member 26 may be disposed on a track 16. The track 16 may be disposed between the gripping module 2 and the sample aspiration and dispensing module 4.

In some embodiments, to reasonably arrange structures within the system, as described above, the sample aspiration and dispensing module 4 performs movement along the Y-axis on the XOY coordinate system. The gripping module 2 is arranged side by side with the sample aspiration and dispensing module 4 and performs movement along the X-axis and the Y-axis on the XOY coordinate system and can satisfy movement of a gripping mechanism along the Z-axis. Meanwhile, due to coordination of a plurality of actions, to arrange mechanisms more reasonably, the gripping module 2 and the sample aspiration and dispensing module 4 are provided with the transfer assistance module. For example, in the form of the moving member 26 cooperating with the track 16, the track 16 is arranged along the X-axis. To facilitate the operation of the gripping module 2 and the sample aspiration and dispensing module 4, a top of the track 16 along the X-axis direction may have a certain height difference. The moving member 26 may be provided with a plurality of hole positions, usually three hole positions, for placing two reaction cups and one tip, respectively. A side of the moving member 26 may be provided with a transmission mechanism 18 through a vertical slide rod 17. The transmission mechanism 18 performs transmission along the X-axis direction. The slide rod 17 may move along the X-axis direction, and also enables the moving member 26 to move along the X-axis direction on the track 16 under traction of the slide rod 17. A vertical hole matching the moving member 26 is provided on the slide rod 17 to provide a degree of freedom of the moving member 26 along the Y-axis.

In some embodiments, the gripping module 2 includes a three-axis motion mechanism disposed above the working surface 100. A gripping mechanism may be provided at a lower portion of a Z-axis of the three-axis motion mechanism, and the gripping mechanism may be configured to cooperate with the transfer assistance module. The working surface 100 of the gripping mechanism may be provided with the tip plate 3 for placing the tip and the detection preparation module for placing the reaction cup.

In some embodiments, as shown in FIG. 3, the sample aspiration and dispensing module 4 includes a dual-axis motion mechanism disposed above the working surface 100. The dual-axis motion mechanism may be provided with a first liquid aspiration mechanism 19 and a second liquid aspiration mechanism 20. The first liquid aspiration mechanism 19 may be configured to pick up the tip and aspirate a sample liquid from the sample position 15 of the sample loading channel 10 to the reaction cup. The second liquid aspiration mechanism 20 may be configured to aspirate the reagents to the reaction cup. The first liquid aspiration mechanism 19 and the second liquid aspiration mechanism 20 may be configured to cooperate with the transfer assistance module. The second liquid aspiration mechanism 20 may be provided with a reagent storage chamber 21. Dual axes of the dual-axis motion mechanism refer to the Y-axis and the Z-axis.

In some embodiments, as shown in FIG. 3, the reagent storage chamber 21 includes a storage chamber cavity. A rotation mechanism 38 may be provided at a bottom of the storage chamber cavity. A plurality of reagent sets 22 may be radially disposed in the storage chamber cavity. Any of the plurality of reagent sets 22 may include a reagent tube which contains the monoclonal antibody reagent bound with the magnetic microparticles and the monoclonal antibody reagent labeled with the UCP.

In some embodiments, the sample aspiration and dispensing module 4 uses the dual-axis motion mechanism (to be precise, a Y-axis motion mechanism) for transferring the liquid aspiration mechanisms, and uses a Z-axis motion mechanism for liquid aspiration and dispensing of the liquid aspiration mechanisms. The liquid aspiration mechanisms include the first liquid aspiration mechanism 19 and the second liquid aspiration mechanism 20. After the tip is gripped by the gripping module 2 and placed in one of the hole positions of the moving member 26, a liquid aspiration end of the first liquid aspiration mechanism 19 moves downward, connects to the pipette tip, and aspirates the sample liquid. The reagent storage chamber 21 of the second liquid aspiration mechanism 20 may rotate about a central axis. After rotating to a corresponding position, a tip of the second liquid aspiration mechanism 20 may aspirate reagents and add the reagents into the reaction cup. The plurality of reagent sets 22 may include at least the reagent tube which contains the monoclonal antibody reagent bound with the magnetic microparticles and the monoclonal antibody reagent labeled with the UCP.

In some embodiments, since the second liquid aspiration mechanism 20 needs to be cleaned after each reagent dispensing, the second liquid aspiration mechanism 20 is composed of a steel needle structure and a cleaning structure. The first liquid aspiration mechanism 19 performs sample dispensing using the pipette tip, thereby avoiding sample cross-contamination. After each liquid aspiration, the liquid aspiration end detaches from the tip and prepares for a next liquid aspiration.

During the above process, a volume of each reaction cup is generally 1000 uL. Each test item requires an independent reaction cup. Sample dispensing uses air displacement pipetting (ADP) and a disposable tip structure, with a dispensing amount generally being 50-100 uL. Using the tip can effectively avoid sample cross-contamination. Reagent dispensing uses a reagent needle. The steel needle controls a dispensing amount via a plunger pump. The reagents are dispensed twice, with each dispensing volume being 150 uL. After each dispensing, inner and outer walls of the steel needle are cleaned through a cleaning port to avoid reagent contamination. The reagent storage chamber 21 is generally provided with slots for 12 reagent sets 22. The rotation mechanism 38 is provided at the bottom of the storage chamber cavity. The rotation mechanism 38 drives magnetic microparticle reagent tubes of the reagent sets 22 to rotate, ensuring that a suspension state of a magnetic bead solution is maintained when the reagent sets 22 are placed in the storage chamber cavity.

In practical application, a liquid level detection sensor is provided at the liquid aspiration end of the first liquid aspiration mechanism 19. The liquid level detection sensor is capable of automatically identifying a liquid level height and detecting a position below a liquid level height to perform accurate dispensing.

In some embodiments, the gripping module 2 transfers the reaction cup and the tip via the gripping mechanism (e.g., a gripper). After completing dispensing, the gripping module 2 returns to a fixed position, allowing the gripper to perform transferring of the reaction cup from a moving block, a vibration and mixing unit 23, an incubation unit 24, a washing and separation unit 25, to the optical measurement module 5 in sequence. A dual-axis motion mechanism of the gripping module 2 and a structure of the gripping mechanism (e.g., the gripper) connected below the dual-axis motion mechanism are easily understood by those skilled in the art. Those skilled in the art can set the structure according to requirements. A motion range of the dual-axis motion mechanism needs to cover the tip plate 3 where the pipette tips and the reaction cups are placed and the detection preparation module.

In some embodiments, as shown in FIGS. 2-3, the detection preparation module includes the incubation unit 24, the vibration and mixing unit 23, and the washing and separation unit 25. The washing and separation unit 25 may include a rotary disk. The rotary disk may be provided with a position 0 and at least three sets of washing positions. Each set of the at least three sets of washing positions may include a liquid withdrawal position and a liquid injection position. A last liquid injection position may be a pre-excitation position. An outer wall of the rotary disk may be provided with a permanent magnet.

In some embodiments, the incubation unit 24 allows a reaction cup loaded with a sample and a reagent to be incubated therein at a preset temperature (e.g., 37±0.1° C.) according to experimental requirements. The incubation unit 24 may include a carrier provided with a plurality of hole positions (e.g., 60 hole positions), thereby simultaneously supporting incubation of 60 reaction cups. An incubation duration may be controlled by software. An alarm may be triggered immediately when a temperature is abnormal.

In some embodiments, the vibration and mixing unit 23 may be configured to place the reaction cup in or on the vibration and mixing unit 23 for vibration and mixing, including but not limited to before incubation, after incubation, after completion of washing and separation and injection of a pre-excitation solution, etc.

In some embodiments, as shown in FIG. 2, the washing and separation unit 25 is configured to implement washing and separation of magnetic beads and waste liquid recovery. An independent cleaning liquid pump is responsible for injection of a cleaning liquid (at the liquid injection position). Three peristaltic pumps are connected with three waste liquid needles 35 and fixed on a lifting frame 27 for withdrawing a waste liquid from the reaction cup (at the liquid withdrawal position). Finally, injection of a pre-excitation solution in the reaction cup is performed by a pre-excitation solution pump. During the above process, the liquid withdrawal position and the liquid injection position are alternately arranged. A permanent magnet with high magnetic field intensity is provided on the outer wall of the rotary disk to ensure that a complex of the monoclonal antibody reagent bound with the magnetic microparticles, an antigen of the sample, and the monoclonal antibody reagent labeled with the UCP is bound to a wall of the reaction cup. The permanent magnet with the high magnetic field intensity may be purchased by those skilled in the art based on calculated values according to requirements. The arrangement of the lifting frame 27 is a well-known technology in the art. Those skilled in the art may set the lifting frame 27 according to requirements.

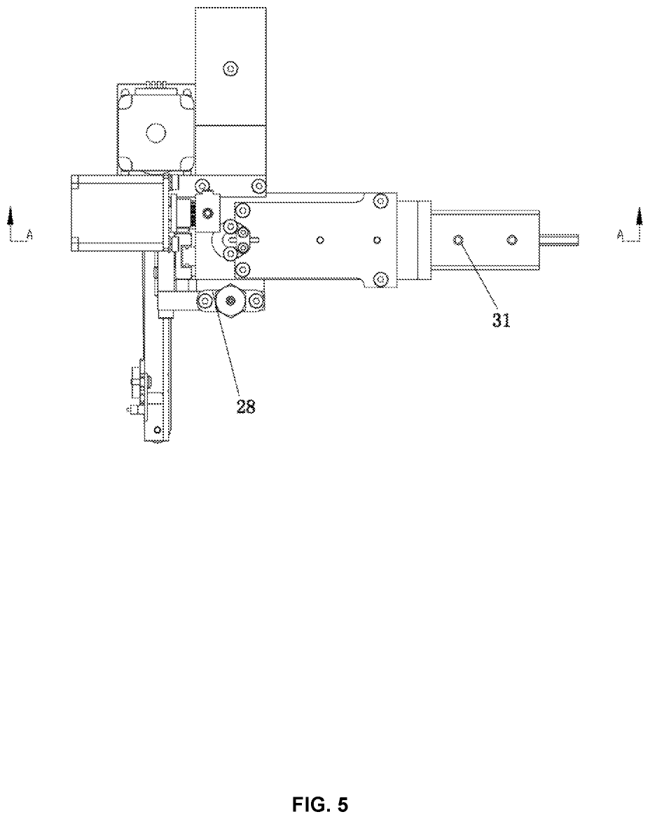

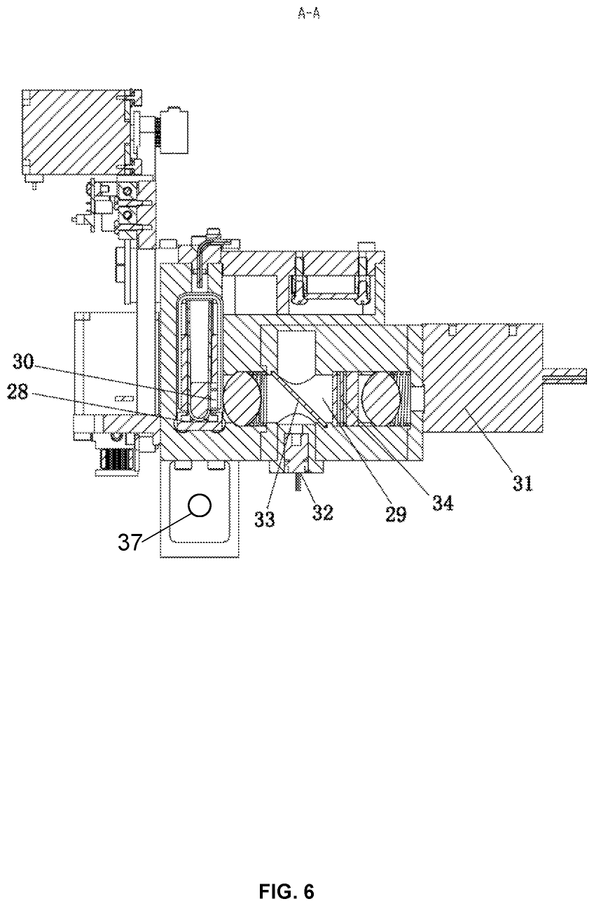

FIG. 5 is a schematic structural diagram of an optical measurement module according to some embodiments of the present disclosure. FIG. 6 is a sectional view along an A-A direction of FIG. 5.

In some embodiments, as shown in FIGS. 5-6, the optical measurement module 5 may include a detection position 28. The detection position 28 may be configured to cooperate with a measurement chamber 29 through an absorption-type movable drawer. A hollowed section 30 may be provided in a portion of the absorption-type movable drawer corresponding to a lower portion of the reaction cup. A photodetector 31 and an excitation light source 32 may be provided in the measurement chamber 29 corresponding to the hollowed section 30. A dichroic beam splitter 33 may be provided between the photodetector 31 and the hollowed section 30. An incident surface of the dichroic beam splitter 33 may face the excitation light source 32. A reflective surface of the dichroic beam splitter 33 may face the hollowed section 30. A transmissive surface of the dichroic beam splitter 33 may face the photodetector 31.

In some embodiments, the detection position 28 of the optical measurement module 5 may have an absorption-type movable drawer structure and cooperate with the measurement chamber 29 through the absorption-type movable drawer. The absorption-type movable drawer may be implemented through an electric control mechanism, which is easily understood by those skilled in the art. Those skilled in the art may set the absorption-type movable drawer according to requirements.

After being washed and mixed, the reaction cup is transferred to the detection position 28. The absorption-type movable drawer closes toward the measurement chamber 29. The excitation light source 32 is activated to excite a sample to be tested (or referred to as an analyte). For example, a high-power 980 nm near-infrared laser is used as the excitation light source 32. Light from the excitation light source 32 is directed to the dichroic beam splitter 33, and reflected toward the analyte in the reaction cup corresponding to the hollowed section 30 to generate excited fluorescence. The excited fluorescence is transmitted through the dichroic beam splitter 33 and a fluorescence signal of a specific wavelength (e.g., 540 nm) is measured by the photodetector 31. A high-sensitivity photomultiplier tube (PMT) is adopted to detect photon signals generated by chemiluminescence.

In some embodiments, as shown in FIG. 6, to further improve the detection effect, a combination 34 of a polarizer, an interference filter, a color filter, or the like, may be provided in a corresponding channel of the measurement chamber 29.

In some embodiments, after measurement is completed, the gripping module 2 transfers the reaction cup to a waste liquid recovery position to discard the reaction cup or to withdraw waste liquid first and then discard the reaction cup.

In some embodiments, a detection method of the system for upconversion chemiluminescence detection based on the magnetic microparticles may include the following operations:

Placing the plurality of samples in the sample rack 9, and introducing the plurality of samples into the system through the sample carrying module 1.

Scanning an identification number of an unprocessed sample tube in a first position, downloading a test item through the identification number, automatically uncapping the sample tube, and during a process of automatically uncapping the sample tube, gripping, by the gripping module 2, the reaction cup and the tip to a preset position.

Loading, by the sample aspiration and dispensing module 4, the pipette tip, aspirating a fixed amount of sample, and adding the fixed amount of sample to the reaction cup; aspirating, by the sample aspiration and dispensing module 4 based on obtained information of the test item, the monoclonal antibody reagent bound with the magnetic microparticles and the monoclonal antibody reagent labeled with the up-converting phosphor, respectively, adding the monoclonal antibody reagent bound with the magnetic microparticles and the monoclonal antibody reagent labeled with the up-converting phosphor to the reaction cup containing the fixed amount of sample, and performing a double-antibody sandwich immunoassay or a competitive immunoassay.

Waiting, by the sample aspiration and dispensing module 4, for a next sample tube 8 to perform sample addition; gripping, by the gripping module 2, the reaction cup to which the fixed amount of sample and the reagents are added, transferring the reaction cup to the detection preparation module, performing shaking and mixing followed by incubating at a constant temperature for a preset time, and performing washing and separation; during a process of washing and separation, adsorbing, by the permanent magnet, the magnetic microparticles to an inner wall of the reaction cup, washing away unbound monoclonal antibody reagent labeled with the up-converting phosphor until only a complex of the monoclonal antibody reagent bound with the magnetic microparticles, an antigen of the fixed amount of sample, and the monoclonal antibody reagent labeled with the up-converting phosphor remains in the reaction cup.

Transferring the reaction cup to the optical measurement module 5, exciting, by the optical measurement module 5 using the excitation light source 2, the up-converting luminescent label, detecting a fluorescence signal intensity at a preset wavelength, and analyzing and calculating a concentration of an analyte based on the fluorescence signal intensity.

During implementation of the embodiments of the present disclosure, samples in the sample tube are placed in the sample rack 9, pass through an input conveyor belt along the X-axis and an input queue area along the Y-axis until reaching a moving frame traction area. The moving frame 12 causes the sample rack 9 to drive the sample tube 8 to roll by the frictional rolling of the rollers 13 and continuously scan the identification number of the sample tube 8. The test item is downloaded through the identification number. The sample tube 8 is uncapped. An air displacement pipettor (ADP) module of the first liquid aspiration mechanism 19 loads the pipette tip, automatically detects a liquid level and inserts into the liquid level, aspirates a certain amount of sample, and adds the certain amount of sample to the reaction cup. The sample tube 8 is capped and moved out of the sample position, waiting for dispensing of a next sample tube 8. The second liquid aspiration mechanism 20 aspirates the monoclonal antibody reagent bound with the magnetic microparticles and the monoclonal antibody reagent labeled with the up-converting phosphor according to the information of the test item and adds the reagents to the reaction cup already containing the sample to perform the double-antibody sandwich immunoassay or the competitive immunoassay. Then the gripping module 2 grips the reaction cup, transfers the reaction cup to the vibration and mixing unit 23, performs high-speed shaking, transfers the reaction cup to the incubation unit 24, incubates at a constant temperature for 10 minutes, and then enters the washing and separation unit 25. The permanent magnet adsorbs the magnetic beads to the wall of the reaction cup. The monoclonal antibody reagent labeled with the UCP that is not bound with the magnetic microparticles is washed away. Only a complex to be tested remains in the reaction cup. After a certain amount of diluent is added, the reaction cup is transferred to the optical measurement module 5. The optical measurement module 5 excites up-converting fluorescence through a high-power 980 nm near-infrared laser, and detects a fluorescence signal of a 540 nm wavelength, thereby analyzing and calculating the concentration of the analyte.

It is understood that the UCP can be repeatedly excited by an external near-infrared 980 nm laser, and an emitted fluorescence signal is stable without phenomena such as photobleaching. However, in chemiluminescent systems, since the particle size of magnetic beads is generally much larger than the volume of antibody proteins and fluorescent labels, a certain degree of fluorescence signal shielding occurs, resulting in poor repeatability of signal detection. In particular, luminescence based on acridinium ester biochemical reaction technology can only be triggered once by the initiation solution, and the shielding effect of magnetic beads on the luminescence signal is more random, making it difficult to improve the repeatability of multiple measurements. That is, the UCP in the detection method provided by the embodiments of the present disclosure can be repeatedly excited a plurality of times. The excitation energy of a high-power laser is far higher than the biological energy of bioluminescence, and the fluorescence signal intensity is significantly enhanced. Moreover, in addition to the UCP, the excitation light source cannot excite fluorescence of other wavelengths from other consumables and optical device materials used in the detection reaction. Therefore, the background fluorescence signal is small, and the detection sensitivity is high.

In some embodiments, after a plurality of cycles of washing and separation of the magnetic microparticles, 300 uL of diluent is added to the reaction cup after a last separation. An initial mixture containing the magnetic beads and the up-converting luminescent label enters the optical measurement module. After laser excitation, a detected fluorescence signal is recorded as X₁. A target concentration Y₁ is obtained by calculating a concentration of an analyte through a standard curve.

In some embodiments, a standard curve for converting a fluorescence signal to a target concentration is a four-parameter fitting algorithm. Related parameters and a fitting curve of the four-parameter fitting algorithm are pre-obtained through standard solutions, based on which,

100 uL of buffer solution is pumped into the reaction cup through a liquid addition plunger pump to dilute a reaction system to ¾ of an original concentration. A fluorescence signal X₂ is measured again, and a target concentration Y₂ is calculated.

Then 100 uL of buffer solution is pumped into the reaction cup through the liquid addition plunger pump to dilute the reaction system to ⅗ of the original concentration. A fluorescence signal X₃ is measured again, and a target concentration Y₃ is calculated.

Then 100 uL of buffer solution is pumped into the reaction cup through the liquid addition plunger pump to dilute the reaction system to ½ of the original concentration. A fluorescence signal X₄ is measured again, and a target concentration Y₄ is calculated.

Finally, 200 uL of buffer solution is pumped into the reaction cup through the liquid addition plunger pump to dilute the reaction system to ⅜ of the original concentration. A fluorescence signal X₅ is measured again, and a target concentration Y₅ is calculated.

Given a target concentration value as Y, a least squares formula is established: Z=(Y−Y₁)²+(0.75×Y−Y₂)²+(0.6×Y−Y₃)²+(0.5×Y−Y₄)²+(0.375×Y−Y₅)².

A Y value is obtained via the least squares formula such that a Z value is a minimum value. Y is an optimal target concentration value of the analyte after eliminating a magnetic bead shielding effect. More descriptions regarding the optimal target concentration value of the analyte may be found in related descriptions later.

During implementation, adding the buffer solution multiple times can be directly implemented in a measurement area of the optical measurement module 5. The embodiments of the present disclosure perform cascade stepwise dilution of the concentration of the analyte by adding the buffer solution multiple times. The newly added buffer solution disturbs an orientation of the magnetic beads in an original system. Fluorescence signal intensities of different dilution concentrations are obtained based on a plurality of detections, and the optimal target concentration value of the analyte is then calculated through the least squares formula. Since the buffer solution added during the plurality of detections disturbs the orientation of the magnetic beads, fluorescence signal values obtained from a plurality of measurements have multi-directional signal characteristics. More uniform fluorescence signal intensity can be obtained, thereby solving the problem of fluorescence signal shielding of the magnetic beads, and improving repeatability.

In some embodiments, as shown in FIGS. 3-4, the system further includes a processor 36. The sample aspiration and dispensing module 4 may include a fully automatic syringe pump 39 with a preset precision. The processor 36 may be configured to: obtain a plurality of fluorescence signal intensities obtained in a current detection; generate a response slope between the plurality of fluorescence signal intensities and a plurality of concentrations of the analyte based on the plurality of fluorescence signal intensities; and in response to the response slope being within an inconsistent interval, control the fully automatic syringe pump to dynamically change a count of steps to adjust a dynamic dilution ratio for a next detection.

The fully automatic syringe pump 39 refers to a precision plunger pump driven by a high-resolution stepping motor. A step resolution of the fully automatic syringe pump 39 reaches a microliter (uL) level. The fully automatic syringe pump 39 is capable of performing non-equal and variable-step liquid dispensing according to an instruction from the processor 36. In some embodiments, the step resolution of the fully automatic syringe pump 39 may be preset by a person skilled in the art according to actual needs.

The processor 36 may process data and/or information obtained from other components of the system. The processor 36 may execute program instructions based on the data, the information, and/or processing results to perform one or more functions described in the present disclosure. In some embodiments, the processor 36 may include one or more sub-processing devices (e.g., a single-core processing device or a multi-core multi-chip processing device). Merely by way of example, the processor 36 may include a central processing unit (CPU), an application-specific instruction processor (ASIP), a controller, a microcontroller unit, a microprocessor, or any combination thereof. In some embodiments, the processor 36 may be integrated within the system. In some embodiments, the processor 36 may be a remote server.

The fluorescence signal intensity refers to a quantified electrical signal value that is captured and converted by a photodetector (e.g., a photomultiplier tube) in the optical measurement module 5 when the UCP is excited by a pulsed laser at a specific wavelength. In some embodiments, the processor 36 may obtain the fluorescence signal intensities through the optical measurement module 5. More descriptions regarding the fluorescence signal intensities may be found in the related descriptions above.

The response slope refers to an instantaneous rate of change of the fluorescence signal intensity collected by the optical measurement module 5 with respect to a change in the concentration of the analyte. The concentration of the analyte is a sample concentration, which is also referred to as a theoretical concentration of the analyte. The concentration of the analyte may be obtained by querying a standard curve.

In some embodiments, the processor 36 may generate a slope vector at different dilution stages as the response slope by calculating a ratio of a difference between fluorescence signal intensities of adjacent dilution nodes to a theoretical dilution gradient based on the plurality of fluorescence signal intensities. The theoretical dilution gradient refers to a difference between preset dilution ratios of the adjacent dilution nodes. Merely by way of example, if an original concentration of the analyte before dilution is 1, a detected fluorescence signal intensity is Q1 when a first dilution is performed at a preset dilution ratio of 0.6; and a detected fluorescence signal intensity is Q2 when a second dilution is performed at a preset dilution ratio of 0.5, then 0.6 and 0.5 are the adjacent dilution nodes in a dilution process. The theoretical dilution gradient is 0.6-0.5=0.1; and the response slope=(Q2−Q1)/0.1.

The inconsistent interval refers to a photon shielding region caused by an excessively high distribution density of magnetic beads. At this time, photons are partially blocked by the magnetic beads, causing a measured fluorescence signal intensity to be far lower than a theoretical value. A consistent interval refers to a concentration range where a distribution density of the magnetic beads has dropped below a critical shielding threshold, such that a mapping logic between the measured fluorescence signal intensity and the concentration of the analyte is close to the standard curve.

In some embodiments, the processor 36 may determine whether the response slope is within the inconsistent interval by comparing the response slope with a preset deviation threshold. For example, if the response slope exceeds the preset deviation threshold, it is determined that the response slope is within the inconsistent interval. The preset deviation threshold may be preset by a person skilled in the art. For example, the preset deviation threshold may be that the response slope deviates from a slope at a corresponding position of the standard curve by 20%.

The dynamic dilution ratio refers to a dilution ratio that can be dynamically adjusted and optimized. In some embodiments, the dynamic dilution ratio may be determined based on a deviation magnitude of the response slope from a slope at a corresponding position of the standard curve. A larger deviation magnitude corresponds to a larger adjustment magnitude of the dynamic dilution ratio. For example,

$$J = \frac{j}{exp ( \frac{K - k}{k} )} ,$$

where J denotes the dynamic dilution ratio; j denotes the preset dilution ratio; K denotes the response slope; and k denotes the slope at the corresponding position of the standard curve.

In some embodiments, the preset dilution ratio may be preset by a person skilled in the art, and the person skilled in the art may preset a maximum value of the dynamic dilution ratio.

In some embodiments, in response to a determination that the response slope is within the inconsistent interval, the processor 36 may control the fully automatic syringe pump to dynamically change a count of steps through a preset program to adjust the dynamic dilution ratio for a next detection. The preset program may be preset by a person skilled in the art. The processor may determine the count of steps of the fully automatic syringe pump by querying a preset table based on the dynamic dilution ratio. The preset table is configured to characterize a correspondence relationship between the count of steps of the fully automatic syringe pump and the dynamic dilution ratio. In some embodiments, the preset table may be constructed based on historical data.

In some embodiments of the present disclosure, through real-time monitoring and analysis of the response slope by the processor, the system can autonomously identify whether a current reaction system is within the inconsistent interval with severe magnetic bead shielding, and drive the high-precision fully automatic syringe pump to dynamically adjust the count of steps for the next operation, thereby adjusting the dynamic dilution ratio for the next detection. The system achieves a shift from blind fixed-gradient dilution to on-demand precise step dilution, significantly reducing measurement time and dilution cycles required for a high-concentration sample to approach the consistent interval, and improving detection precision. Meanwhile, the system avoids signal distortion caused by physical shielding from an algorithmic source, improving the accuracy of results.

In some embodiments, the processor 36 is further configured to: after the first liquid aspiration mechanism 19 completes an N-th proportional dilution, calculate a dilution coefficient; and in response to a difference between the dilution coefficient and a preset dilution ratio of the N-th proportional dilution exceeding a consistency threshold, control the moving member 26 to carry the reaction cup back to a sample position to trigger the first liquid aspiration mechanism 19 to perform a next dilution based on the dynamic dilution ratio. The consistency threshold may be determined through a threshold model based on a magnetic bead feature, a buffer solution feature, and a luminescent material feature. The threshold model may be a machine learning model.

In some embodiments, the first liquid aspiration mechanism 19 may be configured to pick up a tip and aspirate a buffer solution into the reaction cup. More descriptions regarding the first liquid aspiration mechanism may be found in FIG. 3 and the related descriptions thereof.

The dilution coefficient refers to a ratio of a measured concentration of the analyte after completing the N-th proportional dilution to an original concentration of the analyte before dilution. In some embodiments, after the first liquid aspiration mechanism 19 completes the N-th proportional dilution and mixing measurement, the processor 36 may obtain a fluorescence signal intensity of a current dilution node in real time through the optical measurement module 5; inversely derive the measured concentration of the analyte through a built-in standard curve, and calculate a ratio of the measured concentration of the analyte to the original concentration of the analyte to obtain the dilution coefficient.

The consistency threshold refers to a dynamic critical value for determining whether the dilution coefficient enters the consistent interval. It should be noted that the consistency threshold is not a fixed constant, but a dynamic threshold that adaptively adjusts with changes in a detection environment.

In some embodiments, the consistency threshold may be set by a person skilled in the art based on experience.

In some embodiments, the processor 36 may determine the consistency threshold through the threshold model based on the magnetic bead feature, the buffer solution feature, and the luminescent material feature.

The threshold model refers to a model for determining the consistency threshold. In some embodiments, the threshold model is a machine learning model. For example, the threshold model includes one or more combinations of a deep neural network (DNN) model, a convolutional neural network (CNN) model, a custom model, or the like.

In some embodiments, an input of the threshold model includes the magnetic bead feature, the buffer solution feature, and the luminescent material feature, and an output of the threshold model includes the consistency threshold.

The magnetic bead feature may be used to characterize relevant features of the magnetic beads. For example, the magnetic bead feature includes a particle size, an absorbance rate, or the like, of the magnetic beads. In some embodiments, the magnetic bead feature may be input and obtained by a person skilled in the art.

The buffer solution feature is used to characterize relevant features of the buffer solution. For example, the buffer solution feature includes a refractive index, a transparency, or the like, of the buffer solution. In some embodiments, the buffer solution feature may be obtained by a person skilled in the art based on actual detection.

The luminescent material feature is used to characterize relevant features of the UCP. For example, the luminescent material feature includes a fluorescence signal intensity, or the like, of the UCP. More descriptions regarding the fluorescence signal intensity may be found in the related descriptions above.

In some embodiments, the processor 36 may input a plurality of sets of training samples with training labels into an initial threshold model, construct a loss function based on an output of the initial threshold model and the training labels, iteratively update parameters of the initial threshold model based on the loss function, training ends until a preset training condition is satisfied, and a trained threshold model is obtained. The preset training condition may include, but is not limited to, convergence of the loss function, a training epoch reaching a threshold, or the like. An approach of iterative update may include gradient descent or a simulated annealing algorithm, or the like.

The training samples may include a sample magnetic bead feature, a sample buffer solution feature, and a sample luminescent material feature. In some embodiments, the training samples may be constructed based on historical experimental data.

The training labels may include a consistency threshold corresponding to the training sample. In some embodiments, the training labels may be a ratio of a difference between measured concentrations of the analyte of adjacent dilution nodes to a difference between concentrations of the analyte (i.e., theoretical concentrations of the analyte) under conditions of the training samples. The theoretical concentrations of the analyte may be obtained by querying the standard curve. An approach for obtaining the measured concentrations of the analyte may be found in the related descriptions above.

In some embodiments, the processor 36 may determine whether the reaction system after dilution reaches the consistency interval by comparing a magnitude of a difference between the dilution coefficient and the N-th preset dilution ratio. If the magnitude of the difference between the dilution coefficient and the N-th preset dilution ratio exceeds the consistency threshold, the reaction system after dilution does not reach the consistency interval. In this case, the processor 36 may generate a control instruction through the preset program, and control the moving member 26 to carry the reaction cup back to the sample position to trigger the first liquid aspiration mechanism 19 to perform a next dilution based on the dynamic dilution ratio.

In some embodiments of the present disclosure, by introducing the machine learning model, the system can adaptively determine consistency between the measured dilution coefficient and the preset dilution ratio based on multidimensional physical features of the magnetic beads, the buffer solution, and the luminescent material. The system not only eliminates systematic errors caused by physical differences between different batches of reagents, ensuring that only high-quality data entering the consistency interval participates in final concentration calculation, but also controls the moving member to carry the reaction cup back to the sample position for dynamic compensation of dilution, thereby optimizing a path for approaching the consistency interval, and improving detection repeatability and accuracy.

In some embodiments, the processor 36 is further configured to: determine an optimized least squares formula based on a plurality of dynamic dilution ratios and a convergence coefficient. The convergence coefficient may be determined based on a dynamic dilution difference and a consistency difference.

The convergence coefficient refers to a weight factor assigned to each of summation factors in the least squares formula. The convergence coefficient is used to adjust a contribution degree of different summation factors to a final result Y.

In some embodiments, the convergence coefficient may be preset by a person skilled in the art.

The dynamic dilution difference refers to a difference between dynamic dilution ratios of the adjacent dilution nodes. The consistency difference refers to a difference between a difference between the dilution coefficient and the N-th preset dilution ratio and the consistency threshold.

In some embodiments, the processor may determine an initial convergence coefficient for each of the summation factors through a coefficient calculation formula based on the dynamic dilution difference and the consistency difference, and then obtains the convergence coefficient for each of summation factors by performing normalization processing on initial convergence coefficients of all the summation factors. Merely by way of example, the coefficient calculation formula is: μ=μ₀×σ×(1−θ), where μ denotes the initial convergence coefficient; ulo denotes a preset value; σ denotes a reciprocal of the dynamic dilution difference; θ denotes the consistency difference. It is worth noting that when (1−θ) is 0 or approaches 0 (e.g., 0.05), the initial convergence coefficient may be set to the preset value (e.g., 1).

In some embodiments, the processor 36 may correspondingly replace the preset dilution ratios in an original least squares formula with the dynamic dilution ratios, and then correspondingly assign the convergence coefficient to each of the summation factors to obtain the optimized least squares formula. Merely by way of example, Z=H×(Y−Y₁)²+A×(a×Y−Y₂)²+B×(b×Y−Y₃)²+ . . . +M(m×Y−Yₙ)², where H, A, B . . . M denote convergence coefficients; and a, b . . . m denote dynamic dilution ratios.

In some embodiments of the present disclosure, by constructing the optimized least squares formula associated with the dynamic dilution ratios and introducing the convergence coefficient determined based on dual dimensions of the dynamic dilution difference and the consistency difference, the system implements intelligent weighted processing of measurement data points. Moreover, the method can automatically identify and weaken weights of data points subject to greater interference from the shielding effect, and strengthen contributions of high-quality data points within the consistency interval, thereby ensuring that a final output optimal target concentration value can achieve fast and accurate convergence in a complex dynamic dilution process, and enhancing data robustness of the system under wide-range detection.

In some embodiments, the excitation light source 32 is configured to output a pulsed laser. The processor is further configured to: obtain historical signal intensities and historical decay lifetimes; correct a magnetic bead shielding coefficient of the magnetic beads by comparing deviation degrees of the fluorescence decay lifetimes under a plurality of the dynamic dilution ratios; and optimize the dynamic dilution ratio for a next detection based on the magnetic bead shielding coefficient and the response slope.

In some embodiments, the excitation light source may include a pulsed modulation component (not shown in the figures). The pulsed modulation component may be configured to drive the excitation light source 32 to periodically switch on and off, such that the excitation light source 32 outputs the pulsed laser.

The pulsed modulation component refers to a high-frequency electronic control unit integrated in a drive circuit of the excitation light source 32. In some embodiments, the processor 36 may send a square wave control signal with a preset duty cycle and frequency to the pulsed modulation component, and the drive circuit rapidly may switch an on-off state of the excitation light source 32 (e.g., a laser diode) according to the square wave control signal, thereby outputting the pulsed laser. The pulsed laser refers to a laser that outputs laser energy intermittently in a pulse form. A pulse period of the pulsed laser may be adjusted based on the fluorescence decay lifetime. For example, a longer fluorescence decay lifetime corresponds to a longer pulse period.

The fluorescence decay lifetime refers to a duration required for a fluorescence signal intensity of an up-converting luminescent label to decay to a preset proportion of an initial value after the up-converting luminescent label is excited. The preset proportion may be preset by a person skilled in the art. For example, the preset proportion is 1/e, or the like.

In some embodiments, the processor 36 may control the optical measurement module 5 to perform high-speed continuous sampling in a microsecond-level time after the pulsed laser is turned off, record a decline trajectory of the fluorescence signal intensity over time, and determine the fluorescence decay lifetime by fitting the decline trajectory.

The deviation degree refers to a degree to which a measured fluorescence decay lifetime deviates from a theoretical decay lifetime. The theoretical decay lifetime may be determined through experiments. In some embodiments, the processor 36 may use a ratio of an absolute value of a difference between the measured fluorescence decay lifetime and the theoretical decay lifetime to the theoretical decay lifetime as the deviation degree of the fluorescence decay lifetime.

The magnetic bead shielding coefficient is a correction parameter for quantifying the hindrance effect of the magnetic beads on the escape of fluorescence photons caused by physical accumulation, reabsorption, or multiple scattering. The magnetic bead shielding coefficient is used to reflect a real absorption capacity of the magnetic beads for fluorescence photons in a current detection.

In some embodiments, the processor 36 may determine corrected magnetic bead shielding coefficient based on the deviation degree through a correction formula. Merely by way of example, p=(1+α)×p₀, where p denotes the corrected magnetic bead shielding coefficient; α denotes the deviation degree of the fluorescence decay lifetime; p₀ denotes an original magnetic bead shielding coefficient. The original magnetic bead shielding coefficient may be determined by looking up a table or from a third party based on a material, a size, or the like, of the magnetic beads.

In some embodiments, the processor 36 may determine an optimized dynamic dilution ratio based on the magnetic bead shielding coefficient and the response slope through an optimization formula. Merely by way of example,

$${J}_{1} = \frac{j}{exp ( \frac{K - k}{k} )} / ( 1 + \overline{p} ) ,$$

wherein J₁ denotes the optimized dynamic dilution ratio; j denotes the preset dilution ratio; K denotes the response slope; k denotes a slope at a corresponding position of the standard curve; p̅ denotes a mean value of variation amplitudes of the magnetic bead shielding coefficients under the plurality of dynamic dilution ratios.

In some embodiments of the present disclosure, a physical compensation mechanism based on a time dimension is constructed by setting the pulse modulation component and performing measurement of the fluorescence decay lifetime. The processor uses the deviation degrees of the fluorescence decay lifetimes under different dilution ratios to reversely correct the magnetic bead shielding coefficient, achieving precise quantification of physical shielding interference that cannot be distinguished by colorimetric methods and intensity methods. The processor dynamically adjusts the dilution path in coordination with the response slope, eliminating result deviations caused by magnetic bead reabsorption and scattering, thereby improving repeatability and accuracy of detection.

In some embodiments, as shown in FIG. 5, the detection position 28 may be provided with a rotating assembly 37. The rotating assembly may be configured to drive the reaction cup to rotate at a preset speed. The processor 36 may be further configured to: obtain a signal intensity sequence of a rotating reaction cup; determine a distribution uniformity of the magnetic beads within the rotating reaction cup based on the signal intensity sequence; and in response to the distribution uniformity exceeding a uniformity threshold, issue an instruction to control a moving member to drive the reaction cup to shake again.

The rotating assembly 37 refers to a rotation drive unit integrated on a base of the detection position 28 of the optical measurement module 5. For example, the rotating assembly 37 may include a miniature rotary disk, or the like. In some embodiments, the reaction cup may be placed on the rotating assembly 37. The processor 36 may control an electric control mechanism to drive the rotating assembly to rotate, causing the reaction cup to rotate at the preset speed. The electric control mechanism may be a motor, such as a stepper motor or a brushless DC motor, or the like. By adjusting a pulse frequency of the motor, the reaction cup can achieve smooth circumferential motion at the detection position 28, thereby converting static point excitation of the excitation light source 32 into dynamic circumferential scanning. The preset speed may be determined by a person skilled in the art according to actual needs.

The signal intensity sequence refers to a sequence composed of a plurality of fluorescence signal intensities obtained by continuously triggering a photodetector to sample at a preset frequency for at least one rotation of the reaction cup. It can be understood that since a position of the laser relative to a bottom of the reaction cup continuously changes, the signal intensity sequence actually records luminescence conditions of the magnetic beads at different positions along a circumferential path on the bottom of the cup. In some embodiments, the signal intensity sequence may be obtained by scanning with the photodetector 31.

The distribution uniformity is a physical parameter that quantifies a state of aggregation or sedimentation of the magnetic beads in the reaction cup. A larger distribution uniformity indicates a more uneven distribution of the magnetic beads in the reaction cup.

The processor 36 may determine the distribution uniformity of the magnetic beads in a plurality of ways. In some embodiments, the processor 36 may determine the distribution uniformity of the magnetic beads by calculating a standard deviation of the signal intensity sequence. For example, a smaller standard deviation indicates a more uniform distribution of magnetic beads, and a smaller distribution uniformity of the magnetic beads. In some embodiments, the processor 36 may determine the distribution uniformity of the magnetic beads by calculating a coefficient of variation of the signal intensity sequence. The coefficient of variation is a ratio of the standard deviation to a mean value. It can be understood that if there is agglomeration or uneven settling of the magnetic beads, the plurality of fluorescence signal intensities exhibit significant peak-valley fluctuations, and the coefficient of variation is relatively large at this time.

In some embodiments, in response to the distribution uniformity exceeding the uniformity threshold, the processor 36 may issue an instruction through a preset program to control the moving member 26 to drive the reaction cup to shake again. The processor 36 may determine the uniformity threshold based on statistical analysis of historical experimental data. For example, the processor 36 may use a maximum uniformity from historical experimental data that allows a finally determined concentration of the analyte to meet a preset requirement as the uniformity threshold. The preset requirement may be a difference between the finally determined concentration of the analyte and a manual analysis result being less than 5%, or the like.

In some embodiments of the present disclosure, by analyzing fluctuation features of a plurality of fluorescence signal intensities obtained from circumferential scanning, the processor can identify physical abnormalities such as agglomeration or uneven settling of the magnetic beads. The processor cooperates with the moving member and a mixing position to achieve self-recovery in case of failure. The system not only effectively avoids random errors caused by aggregation of the magnetic beads in single-point measurement, ensuring that signals involved in calculation have extremely high spatial representativeness, but also enhances robustness of the system to complex biochemical reaction environments, thereby ensuring stability of detection results.

In some embodiments, the processor 36 may be further configured to: determine a fluorescence signal fluctuation rate based on the signal intensity sequence; determine a fluctuation weight for a current detection based on the fluorescence signal fluctuation rate; and determine an optimal target concentration value of an analyte based on the fluctuation weight and the plurality of fluorescence signal intensities.

The fluorescence signal fluctuation rate refers to a statistical indicator configured to quantify a dispersion degree of the plurality of fluorescence signal intensities during rotation. The fluorescence signal fluctuation rate reflects local consistency of a magnetic bead distribution within the reaction cup in a single detection.

In some embodiments, the processor 36 may determine the fluorescence signal fluctuation rate by calculating a coefficient of variation of the signal intensity sequence based on the signal intensity sequence. A larger coefficient of variation indicates more drastic fluctuations in fluorescence signal intensity and a correspondingly higher fluorescence signal fluctuation rate.

The fluctuation weight refers to a trust factor assigned to each of summation factors in a multi-point fitting calculation according to signal stability. In some embodiments, the processor 36 may perform normalization processing on a reciprocal of the fluorescence signal fluctuation rate to obtain the fluctuation weight for the current detection. For example, a lower fluorescence signal fluctuation rate indicates a more stable signal, and an assigned fluctuation weight W approaches 1. A higher fluorescence signal fluctuation rate indicates a greater signal fluctuation, and the assigned fluctuation weight W approaches 0.

In some embodiments, the processor may optimize the least squares formula again based on the fluctuation weight and the plurality of fluorescence signal intensities. The optimal target concentration value of the analyte is reversely calculated through a re-optimized least squares formula. Merely by way of example, Z=H×(Y−Y₁)²+Q₁×A×(a×Y−Y₂)²+Q₂×B×(b×Y−Y₃)²+ . . . +Q₍ₙ₋₁₎×M(m×Y−Yₙ)², where Q₁, Q₂ . . . Q₍ₙ₋₁₎ denote the fluctuation weights. More descriptions regarding the optimal target concentration value of the analyte may be found in the related descriptions above.

According to the embodiments of the present disclosure, intelligent grading of measurement data reliability is achieved by introducing a dynamic weighting mechanism based on the fluorescence signal fluctuation rate. The processor automatically assigns a high weight to stable data points and a low weight to fluctuating data points through real-time evaluation of a dispersion degree of scanning signals, and integrates the weights into a weighted least squares formula for concentration tracing. The system effectively resolves random interference on detection results caused by slight uneven distribution of the magnetic beads, enhances system robustness, reduces ineffective downtime intervention, and ensures that the optimal target concentration of the analyte still maintains high convergence accuracy and confidence under complex physical backgrounds.

The foregoing has described the basic concepts. Obviously, to a person skilled in the art, the foregoing detailed disclosure is merely an example and does not constitute a limitation on the present disclosure. Although not explicitly stated herein, a person skilled in the art may make various modifications, improvements, and corrections to the present disclosure. Such modifications, improvements, and corrections are suggested in the present disclosure. Therefore, such modifications, improvements, and corrections still fall within the spirit and scope of the exemplary embodiments of the present disclosure.

What is claimed is:

1. A system for upconversion chemiluminescence detection based on magnetic microparticles, comprising: a working surface; wherein the working surface includes:
a sample carrying module configured to input, preload, and output a plurality of samples;
a gripping module configured to grip a reaction cup and a pipette tip from a previous working position to a next working position;
a sample aspiration and dispensing module configured to aspirate at least one of the plurality of samples and reagents and dispense the at least one of the plurality of samples and the reagents into the reaction cup, wherein the reagents include at least a monoclonal antibody reagent bound with magnetic microparticles and a monoclonal antibody reagent labeled with up-converting phosphor;
a detection preparation module configured to perform one or more cycles of mixing, incubation, and a plurality of cycles of washing and separation on the at least one of the plurality of samples and the reagents dispensed into the reaction cup; and
an optical measurement module configured to excite an up-converting luminescent label and detect a fluorescence signal intensity.

2. The system according to claim 1, wherein the sample carrying module includes a sample loading channel and a sample output channel; the sample loading channel is provided with a sample inlet for placing a sample rack, the sample output channel is provided with a sample outlet for outputting the sample rack, and the sample loading channel and the sample output channel are respectively provided with a traction unit for the sample rack;
a moving frame is respectively provided at ends of the sample loading channel and the sample output channel for the sample rack, and the moving frame is provided with a moving traction unit;
a cap removal position is provided between the sample loading channel and the sample output channel, and a cap removal mechanism is provided corresponding to the cap removal position;
the sample loading channel is provided with rollers tangent to a sample tube, and a side of the cap removal position is provided with a barcode scanning mechanism for the sample tube;
the sample loading channel is provided with a sample position, and the sample position is configured to cooperate with the sample aspiration and dispensing module.

3. The system according to claim 1, wherein the gripping module and the sample aspiration and dispensing module are provided with a transfer assistance module, and the transfer assistance module is configured to perform transfer assistance for a travel path between the gripping module and the sample aspiration and dispensing module;
the transfer assistance module includes a moving member provided with a plurality of hole positions, the moving member is disposed on a track, and the track is disposed between the gripping module and the sample aspiration and dispensing module.

4. The system according to claim 3, wherein the gripping module includes a three-axis motion mechanism disposed above the working surface, a gripping mechanism is provided at a lower portion of a Z-axis of the three-axis motion mechanism, and the gripping mechanism is configured to cooperate with the transfer assistance module; the working surface of the gripping mechanism is provided with a tip plate for placing the pipette tip and the detection preparation module for placing the reaction cup.

5. The system according to claim 3, wherein the sample aspiration and dispensing module includes a dual-axis motion mechanism disposed above the working surface, the dual-axis motion mechanism is provided with a first liquid aspiration mechanism and a second liquid aspiration mechanism, the first liquid aspiration mechanism is configured to pick up the pipette tip and aspirate a sample liquid from the sample position of the sample loading channel to the reaction cup, the second liquid aspiration mechanism is configured to aspirate the reagents to the reaction cup, the first liquid aspiration mechanism and the second liquid aspiration mechanism are configured to cooperate with the transfer assistance module, and the second liquid aspiration mechanism is provided with a reagent storage chamber.

6. The system according to claim 5, wherein the reagent storage chamber includes a storage chamber cavity, a rotation mechanism is provided at a bottom of the storage chamber cavity, and a plurality of reagent sets are radially disposed in the storage chamber cavity; any of the plurality of reagent sets includes a reagent tube which contains the monoclonal antibody reagent bound with the magnetic microparticles and the monoclonal antibody reagent labeled with the up-converting phosphor.

7. The system according to claim 1, wherein the detection preparation module includes an incubation unit, a vibration and mixing unit, and a washing and separation unit; the washing and separation unit includes a rotary disk, the rotary disk is provided with a position 0 and at least three sets of washing positions, each set of the at least three sets of washing positions includes a liquid withdrawal position and a liquid injection position, and a last liquid injection position is a pre-excitation position; an outer wall of the rotary disk is provided with a permanent magnet.

8. The system according to claim 1, wherein the optical measurement module includes a detection position, the detection position is configured to cooperate with a measurement chamber through an absorption-type movable drawer, a hollowed section is provided in a portion of the absorption-type movable drawer corresponding to a lower portion of the reaction cup, a photodetector and an excitation light source are provided in the measurement chamber corresponding to the hollowed section, a dichroic beam splitter is provided between the photodetector and the hollowed section, an incident surface of the dichroic beam splitter faces the excitation light source, a reflective surface of the dichroic beam splitter faces the hollowed section, and a transmissive surface of the dichroic beam splitter faces the photodetector.

9. The system according to claim 1, wherein a detection method of the system comprises the following operations:
placing the plurality of samples in a sample rack, and introducing the plurality of samples into the system through the sample carrying module;
scanning a sample tube identification number of an unprocessed sample tube in a first position, downloading a test item through the sample tube identification number, automatically uncapping the sample tube, and during a process of automatically uncapping the sample tube, gripping, by the gripping module, the reaction cup and the pipette tip to a preset position;
loading, by the sample aspiration and dispensing module, the pipette tip, aspirating a fixed amount of sample, and adding the fixed amount of sample to the reaction cup; aspirating, by the sample aspiration and dispensing module based on obtained information of the test item, the monoclonal antibody reagent bound with the magnetic microparticles and the monoclonal antibody reagent labeled with the up-converting phosphor, respectively, adding the monoclonal antibody reagent bound with the magnetic microparticles and the monoclonal antibody reagent labeled with the up-converting phosphor to the reaction cup containing the fixed amount of sample, and performing a double-antibody sandwich immunoassay or a competitive immunoassay;
waiting, by the sample aspiration and dispensing module, for a next sample tube to perform sample addition; gripping, by the gripping module, the reaction cup to which the fixed amount of sample and the reagent are added, transferring the reaction cup to the detection preparation module, performing shaking and mixing followed by incubating at a constant temperature for a preset time, and performing washing and separation; during a process of washing and separation, adsorbing, by the permanent magnet, the magnetic microparticles to an inner wall of the reaction cup, washing away unbound monoclonal antibody reagent labeled with the up-converting phosphor until only a complex of the monoclonal antibody reagent bound with the magnetic microparticles, an antigen of the fixed amount of sample, and the monoclonal antibody reagent labeled with the up-converting phosphor remains in the reaction cup; and
transferring the reaction cup to the optical measurement module, exciting, by the optical measurement module using the excitation light source, the up-converting luminescent label, detecting a fluorescence signal intensity at a preset wavelength, and analyzing and calculating a concentration of an analyte based on the fluorescence signal intensity.

10. The system according to claim 9, wherein after a plurality of cycles of washing and separation are completed based on the magnetic microparticles, a preset amount of diluent is added to the reaction cup after last separation, an initial mixture containing magnetic beads and the up-converting luminescent label is introduced into the optical measurement module, the initial mixture is excited through a laser, a fluorescence signal is detected recorded as X₁, the concentration of the analyte is calculated through a standard curve to obtain a target concentration Y₁;
a preset amount of buffer solution is pumped into the reaction cup to dilute a reaction system to a concentration a₁, a fluorescence signal X₂ is measured again, and a target concentration Y₂ is calculated;
the preset amount of buffer solution is pumped into the reaction cup again to dilute the reaction system to a concentration a₂ of an original concentration, a fluorescence signal X₃ is measured again, and a target concentration Y₃ is calculated;
the preset amount of buffer solution is pumped into the reaction cup again to dilute the reaction system to a concentration a₃ of the original concentration, a fluorescence signal X₄ is measured again, and a target concentration Y₄ is calculated;
the preset amount of buffer solution is pumped into the reaction cup again to dilute the reaction system to a concentration a₄ of the original concentration, a fluorescence signal X₅ is measured again, and a target concentration Y₅ is calculated;
given a target concentration value Y, a least squares formula is established, Z=(Y−Y₁)²+(a₁×Y−Y₂)²+(a₂×Y−Y₃)²+(a₃×Y−Y₄)²+(a₄×Y−Y₅)²; and
a Y value is obtained via the least squares formula such that a Z value is a minimum value, wherein Y is an optimal target concentration value of the analyte after eliminating a magnetic bead shielding effect.

11. The system according to claim 1, further comprising a processor, wherein the sample aspiration and dispensing module includes a fully automatic syringe pump with a preset precision;
the processor is configured to: obtain a plurality of fluorescence signal intensities obtained in a current detection; generate a response slope between the plurality of fluorescence signal intensities and a plurality of concentrations of the analyte based on the plurality of fluorescence signal intensities; and in response to the response slope being within an inconsistent interval, control the fully automatic syringe pump to dynamically change a count of steps to adjust a dynamic dilution ratio for a next detection.

12. The system according to claim 11, wherein the processor is further configured to: after a first liquid aspiration mechanism completes an N-th proportional dilution, calculate a dilution coefficient; and in response to a difference between the dilution coefficient and a preset dilution ratio of the N-th proportional dilution exceeding a consistency threshold, control a moving member to carry the reaction cup back to a sample position to trigger the first liquid aspiration mechanism to perform a next dilution based on the dynamic dilution ratio; wherein the consistency threshold is determined through a threshold model based on a magnetic bead feature, a buffer solution feature, and a luminescent material feature; and the threshold model is a machine learning model.

13. The system according to claim 12, wherein the processor is further configured to:
determine an optimized least squares formula based on a plurality of dynamic dilution ratios and a convergence coefficient; wherein the convergence coefficient is determined based on a dynamic dilution difference and a consistency difference.

14. The system according to claim 11, wherein the excitation light source is further configured to output a pulsed laser;
the processor is further configured to: obtain a fluorescence decay lifetime; correct a magnetic bead shielding coefficient of magnetic beads by comparing deviation degrees of fluorescence decay lifetimes under a plurality of dynamic dilution ratios; and optimize the dynamic dilution ratio for the next detection based on the magnetic bead shielding coefficient and the response slope.

15. The system according to claim 14, wherein a rotating assembly is provided at a detection position, and the rotating assembly is configured to drive the reaction cup to rotate at a preset speed;
the processor is further configured to: obtain a signal intensity sequence of a rotating reaction cup; determine a distribution uniformity of the magnetic beads within the rotating reaction cup based on the signal intensity sequence; and in response to the distribution uniformity exceeding a uniformity threshold, issue an instruction to control a moving member to drive the reaction cup to shake again.

16. The system according to claim 15, wherein the processor is further configured to:
determine a fluorescence signal fluctuation rate based on the signal intensity sequence; determine a fluctuation weight for the current detection based on the fluorescence signal fluctuation rate; and determine an optimal target concentration value of the analyte based on the fluctuation weight and the plurality of fluorescence signal intensities.
